(19) United States
(12) Patent Application Publication
(10) Pub. No.: US 2026/0243743 A1
(43) Pub. Date: Aug. 20, 2026

(54) GAS SENSOR WITH INTEGRATED PRECONCENTRATOR

(71) Applicant: UIF (University Industry Foundation), Yonsei University, Seoul (KR)

(72) Inventors: Jongbaeg KIM, Goyang-si (KR); Jeonghyeop SON, Seoul (KR); Hee-Jin KO, Seoul (KR); Gyu Jun CHOI, Seoul (KR)

(73) Assignee: UIF (University Industry Foundation), Yonsei University, Seoul (KR)

(21) Appl. No.: 19/436,173

(22) Filed: Dec. 30, 2025

(30) Foreign Application Priority Data
Feb. 19, 2025 (KR) 10-2025-0021827

(51) Int. Cl. G01N 33/00

(52) U.S. Cl. CPC G01N 33/0019

(57) ABSTRACT

A gas sensor with an integrated preconcentrator is disclosed. The gas sensor may comprise: a preconcentration unit comprising a material that adsorbs a predetermined target gas, and configured to adsorb the target gas under predetermined adsorption conditions and/or release the adsorbed target gas under predetermined release conditions; a sensing unit adjacent to the preconcentration unit and configured to sense at least the target gas released by the preconcentration unit; and a preconcentration control unit configured to control satisfaction or non-satisfaction of the adsorption conditions and/or the release conditions.

### CROSS-REFERENCE TO RELATED APPLICATION

This application claims priority from Korean Patent Application No. 10-2025-0021827, filed on February 19, 2025, in the Korean Intellectual Property Office, and all the benefits accruing therefrom under 35 U.S.C. 119. The contents of the above application are incorporated herein in their entirety by reference.

### BACKGROUND

### Field

The present invention relates to a gas sensor integrated with a preconcentrator. More specifically, the present invention relates to the structure of a gas sensor including a gas preconcentrator for effective gas detection and a method of fabricating the same. This innovative technology provides an integrated system capable of efficiently detecting and analyzing low-concentration gases.

### Description of Related Art

A gas sensor is a device capable of detecting a specific gas and analyzing its concentration, and can be utilized in various fields such as environmental monitoring, industrial safety, and medical diagnosis. Such sensors operate based on physical or chemical properties of gases and are generally fabricated using metal oxides, organic materials, or nanomaterials. In particular, technology for accurately detecting low-concentration gases can play an important role in applications such as air quality management, chemical leak detection, and biosignal analysis. However, conventional gas sensors often exhibit limitations in detection sensitivity and detection limits.

The problem with conventional gas sensors lies in their inability to effectively detect gases at low concentrations or their lack of selectivity for specific gases. This may result from limitations of the sensing material itself and the absence of collection and concentration functions. Additionally, problems exist where sensors are large in size or have low energy efficiency, resulting in poor portability and complex installation and operation. These factors hinder the practicality and economic efficiency of sensors and make them difficult to be widely used in various industries and application fields.

Therefore, as a technology to solve these problems, development of a new gas sensor technology that integrates collection and detection is required. This technology has the potential to develop in a direction that simplifies existing complex structures while enhancing the concentration of low-concentration gases and detection sensitivity. In particular, it can be designed in a direction that strengthens selectivity for specific gases through combinations of various preconcentration materials and sensing materials, and achieves improvements in terms of energy efficiency and miniaturization.

Although various approaches exist for gas sensor platform design, the present invention presents a design based on MEMS (Micro-Electro-Mechanical Systems) chips. Utilizing MEMS technology enables miniaturization and high performance, and can provide the advantage of reducing power consumption. Conventional gas detection methods utilizing preconcentrators consisted of a preconcentrator device and a gas sensor configured as separate devices connected in series. Such an approach can complicate the system structure and create problems that reduce overall efficiency. The present invention proposes a new approach to overcome these limitations.

### SUMMARY

The problem to be solved by the present invention is to implement a sensor capable of effectively detecting low-concentration gases while operating stably under various environmental conditions. To this end, a design is required that can simplify yet efficiently perform the process of concentrating and detecting gases by integrating a preconcentration unit and a sensing unit.

In one aspect, the present invention provides a gas sensor comprising: a preconcentration unit comprising a material that adsorbs a predetermined target gas, and configured to adsorb the target gas under predetermined adsorption conditions and/or release the adsorbed target gas under predetermined release conditions; a sensing unit adjacent to the preconcentration unit and configured to sense at least the target gas released by the preconcentration unit; and a preconcentration control unit configured to control satisfaction or non-satisfaction of the adsorption conditions and/or the release conditions.

In one embodiment, the adsorption conditions or the release conditions may comprise one or more selected from temperature, pressure, light, and pH.

In one embodiment, the adsorption conditions or the release conditions may be temperature.

In one embodiment, the preconcentration control unit may adsorb or release the target gas by changing the temperature of the preconcentration unit.

In one embodiment, the gas sensor may further comprise a control unit that is at least communicatively coupled to the preconcentration unit, the sensing unit, and the preconcentration control unit, and controls the preconcentration control unit to satisfy or not satisfy the release conditions under predetermined control conditions.

In one embodiment, the control conditions may comprise elapse of a predetermined period of time.

In one embodiment, the preconcentration unit or the sensing unit may comprise one or more selected from: porous polymers or composites thereof including Tenax TA and Tenax GR; carbon allotropes or conductive nanomaterials selected from aligned or non-aligned carbon nanotubes (CNT), graphene, graphene oxide, activated carbon, and activated carbon fiber (ACF); metal-organic frameworks (MOF) or covalent organic frameworks (COF) including ZIF-8, ZIF-67, UiO-66, and MIL series; and metal oxide-based materials selected from SnO₂, ZnO, TiO₂, and WO₃.

In one embodiment, the gas sensor may further comprise a signal analysis unit that is at least communicatively coupled to the sensing unit and configured to analyze signals generated by the sensing unit.

In another aspect, the present invention provides a low-concentration gas sensing method comprising: providing a gas sensor comprising: a preconcentration unit comprising a material that adsorbs a predetermined target gas, and configured to adsorb the target gas under predetermined adsorption conditions and/or release the adsorbed target gas under predetermined release conditions; a sensing unit adjacent to the preconcentration unit and configured to sense at least the target gas released by the preconcentration unit; and a preconcentration control unit configured to control satisfaction or non-satisfaction of the adsorption conditions and/or the release conditions; and sensing, through the sensing unit, the target gas that the preconcentration unit releases after adsorption.

In one embodiment, the adsorption conditions or the release conditions may be provided to comprise one or more selected from temperature, pressure, light, and pH.

In one embodiment, the adsorption conditions or the release conditions may be temperature.

In one embodiment, the preconcentration control unit may be configured to adsorb or release the target gas by changing the temperature of the preconcentration unit.

In one embodiment, the gas sensor may be provided to further comprise a control unit that is at least communicatively coupled to the preconcentration unit, the sensing unit, and the preconcentration control unit, and controls the preconcentration control unit to satisfy or not satisfy the adsorption conditions and/or the release conditions under predetermined control conditions.

In one embodiment, the control unit may be provided such that the control conditions are satisfied when a predetermined period of time elapses.

In one embodiment, the preconcentration unit or the sensing unit may be provided to comprise one or more selected from: porous polymers or composites thereof including Tenax TA and Tenax GR; carbon allotropes or conductive nanomaterials selected from aligned or non-aligned carbon nanotubes (CNT), graphene, graphene oxide, activated carbon, and activated carbon fiber (ACF); metal-organic frameworks (MOF) or covalent organic frameworks (COF) including ZIF-8, ZIF-67, UiO-66, and MIL series; and metal oxide-based materials selected from SnO₂, ZnO, TiO₂, and WO₃.

In one embodiment, the gas sensor may be provided to further comprise a signal analysis unit that is at least communicatively coupled to the sensing unit and configured to analyze signals generated by the sensing unit.

In yet another aspect, the present invention provides a gas monitoring device comprising: a gas sensor comprising: a preconcentration unit comprising a material that adsorbs a predetermined target gas, and configured to adsorb the target gas under predetermined adsorption conditions and/or release the adsorbed target gas under predetermined release conditions; a sensing unit adjacent to the preconcentration unit and configured to sense at least the target gas released by the preconcentration unit; and a preconcentration control unit configured to control satisfaction or non-satisfaction of the adsorption conditions and/or the release conditions; and a signal generation unit configured to generate a signal when the gas sensor detects the target gas at or above a predetermined level.

In one embodiment, the gas monitoring device may be an air pollution detection device, a hazardous gas detection device, an industrial emission detection device, an indoor air quality monitoring device, a chemical plant gas leak alarm device, a household CO₂ monitoring device, a portable radon detector, a medical breath analysis device, an agricultural greenhouse gas detection device, an atmospheric methane detection device, a vehicle exhaust gas detection device, a refinery process gas analysis device, a semiconductor manufacturing process gas monitoring device, a wastewater treatment process gas detection device, a food storage ethylene detection device, a cold storage ammonia detection device, a fire detection smoke analysis device, a radioactive leak detection device, a building carbon dioxide concentration measurement device, an oxygen deficiency alarm device, an anesthetic gas concentration monitoring device, a precision gas chromatography device, a gas fuel mixture ratio measurement device, an offshore oil platform gas detection device, a military chemical warfare response gas detector, a spacecraft internal air quality monitoring device, a natural gas distribution network detection device, an underground mine methane detection device, an electric vehicle battery overheating detection device, a laboratory toxic gas monitoring device, a refrigerant leak detection device, a plasma process gas detection device, an incubator internal oxygen and CO₂ concentration detection device, a power plant exhaust gas detection device, a hydrogen fuel cell system hydrogen leak detection device, an earthquake precursor gas detection device, a city gas supply network detection device, a medical helium gas concentration detection device, a high-altitude oxygen concentration monitoring device, a smart building VOC detection device, a crop storage facility carbon dioxide detection device, a hospital waste incinerator gas monitoring device, an aircraft cabin air quality monitoring device, a combustion process NOx and SOx detection device, an energy plant gas detection device, a subway tunnel air quality detection device, an undersea gas leak detection device, a smart city environmental sensor network, or a biological laboratory gas monitoring device.

The effects of the present invention present the possibility of more precisely measuring gas concentrations in various environments. This technology can contribute to detecting trace amounts of gas that are difficult to detect with conventional gas sensors or improving selectivity for specific gases. Additionally, through an integrated structure, it can help improve miniaturization and portability of sensors and reduce energy consumption. This can open up possibilities for strengthening safety at industrial sites or increasing the efficiency of environmental monitoring systems. Furthermore, in medical or research fields, it can contribute to developing new diagnostic and analytical methods through more sensitive and reliable data collection.

The preconcentrator-integrated gas sensor proposed in the present invention has several distinctive advantages compared to existing research and inventions. First, by integrating the preconcentrator and gas sensor into one device, the structure is simpler and more suitable for miniaturization compared to existing separate systems. This allows effective concentration and detection of low-concentration gases, enabling more accurate measurement of trace amounts of gas that were difficult to detect with conventional sensors. Additionally, by selectively using various preconcentration materials and gas sensing materials, it provides the possibility of fabricating customized sensors for different types of gases. The integrated design of the preconcentrator and sensor can contribute to increasing the energy efficiency of the entire system, shortening detection time, and reducing costs. Moreover, by applying various preconcentrator structures in 3D or 2D forms, it opens up the possibility of implementing optimized designs suitable for application fields.

The integrated gas sensor system presented in the present invention has the possibility of providing several advantages by combining the preconcentrator and sensor into one device. Through this design, miniaturization of the system becomes possible, which can greatly improve portability and ease of installation. Additionally, since the process from gas preconcentration to detection occurs continuously, the overall detection time can be shortened. Integration of the two functions also has the possibility of increasing energy efficiency and realizing cost reduction of the system. Furthermore, this integrated system can contribute to improving the detection capability of low-concentration gases. By having the preconcentrator perform the role of concentrating gases, even trace amounts of gas that are difficult to detect with conventional sensors can be effectively detected. These characteristics can have important significance in various fields such as environmental monitoring, industrial safety, and medical diagnosis.

Another advantage of the present invention lies in its applicability to detect various gases. By appropriately selecting preconcentration materials and gas sensing materials, a system capable of effectively detecting various types of gases can be constructed. This provides the possibility of being particularly useful in industrial sites or research environments where various gases need to be monitored through one platform. In conclusion, the present invention shows the possibility of greatly improving the efficiency and accuracy of gas detection by overcoming the limitations of existing technology through proposing an innovative gas detection system that integrates a gas preconcentrator and sensor. This technology has the potential to play an important role in various fields such as environmental protection, industrial safety, and medical diagnosis, and through further research and development in the future, more advanced integrated gas sensor systems can emerge. This is expected to present a new direction for gas detection and analysis technology.

### BRIEF DESCRIPTION OF DRAWINGS

FIG. 1 is a diagram illustrating the adsorption and desorption process of toluene gas.

FIG. 2 is a diagram illustrating various forms of gas preconcentration materials and the structure of gas sensors utilizing them.

FIG. 3 is a diagram illustrating the structure of a gas sensor platform and the arrangement of preconcentrators and gas sensing materials.

FIG. 4 is a diagram illustrating temperature changes and gas signal changes during one cycle in which the gas sensor of the present invention detects gas.

### DETAILED DESCRIPTIONS

Hereinafter, embodiments of the present invention will be described in detail with reference to the accompanying drawings. Since the present invention may have various changes and may have various forms, specific embodiments are illustrated in the drawings and will be described in detail in the text. However, this is not intended to limit the present invention to the specific disclosed forms, and should be understood to include all modifications, equivalents, and substitutes included in the spirit and technical scope of the present invention. Similar reference numerals have been used for similar components throughout the description of each drawing. In the accompanying drawings, the dimensions of structures are enlarged from actual size for clarity of the present invention.

The terms used in this application are only used to describe specific embodiments and are not intended to limit the present invention. Singular expressions include plural expressions unless the context clearly indicates otherwise. In this application, terms such as "comprise" or "have" are intended to designate the presence of features, numbers, steps, operations, components, or combinations thereof described in the specification, and should be understood not to preclude in advance the possibility of the presence or addition of one or more other features, numbers, steps, operations, components, or combinations thereof. In the context of this specification, terms such as "about" may mean approximately ±1%, approximately ±2%, approximately ±3%, approximately ±4%, approximately ±5%, approximately ±6%, approximately ±7%, approximately ±8%, approximately ±9%, or approximately ±10% of the numerical values described in the specification.

Additionally, descriptions regarding one aspect of the present invention may be applied identically or similarly to identical or similar configurations or terms in descriptions regarding other aspects.

Unless otherwise defined, all terms used herein, including technical or scientific terms, have the same meaning as commonly understood by one of ordinary skill in the art to which the present invention belongs. Terms such as those defined in commonly used dictionaries should be interpreted as having meanings consistent with the meanings they have in the context of the related art, and should not be interpreted in an idealized or overly formal sense unless clearly defined in this application.

A gas sensor according to an embodiment of the present invention may comprise: a preconcentration unit comprising a material that adsorbs a predetermined target gas, and configured to adsorb the target gas under predetermined adsorption conditions and/or release the adsorbed target gas under predetermined release conditions; a sensing unit adjacent to the preconcentration unit and configured to sense at least the target gas released by the preconcentration unit; and a preconcentration control unit configured to control satisfaction or non-satisfaction of the adsorption conditions and/or the release conditions.

In the context of this specification, the meaning of the term "gas sensing" or "gas sensor" may include technology or devices that detect specific gases present in the surrounding environment and measure their concentration. This has the possibility of performing functions such as issuing alarms based on changes in detected gas concentration or collecting and analyzing data. Gas sensing can be utilized in various application fields such as environmental monitoring, industrial safety, and medical diagnosis.

In the context of this specification, the meaning of "target gas" refers to a gas that is the object of adsorption and detection, and may be defined variously depending on the application of the sensor. For example, in industrial safety environments, hazardous gases may be targets, and in environmental monitoring, atmospheric pollutants may be included.

In certain embodiments, the "target gas" is not limited to a specific chemical species and may mean the entire gas mixture present in the space where the sensor is deployed. In this case, the preconcentration unit may be designed to non-selectively adsorb ambient gases, while the sensing unit may be designed to have selective response characteristics for specific gases. Accordingly, it is possible to generally concentrate gases in the environment during the preconcentration stage, and then separately detect only signals corresponding to specific gas components in the sensing unit. This structure can relax constraints on the selectivity of the preconcentration material. Additionally, desired gas information can be extracted through adjustment of the characteristics of the sensing unit even in complex gas environments.

Target gases that the gas sensor of the present invention can sense may include carbon dioxide, methane, ammonia, carbon monoxide, sulfur dioxide, nitrogen oxides, oxygen, radon, hydrogen, volatile organic compounds (VOC), and the like. In addition, in the medical field, acetone or sulfur dioxide gas, and in environmental monitoring, gases related to particulate matter may be potential targets.

Non-limiting examples of the target gases include carbon dioxide (CO₂), methane (CH₄), carbon monoxide (CO), sulfur dioxide (SO₂), nitrogen oxides (NOx), ammonia (NH₃), hydrogen (H₂), oxygen (O₂), radon (Rn), ozone (O₃), acetone, formaldehyde, sulfur trioxide (SO₃), ethylene, propane, butane, xylene, toluene, benzene, acetylene, helium, hydrogen sulfide (H₂S), chlorine (Cl₂), hydrogen fluoride (HF), hydrogen chloride (HCl), bromine (Br₂), chlorine dioxide (ClO₂), nitrogen dioxide (NO₂), argon (Ar), krypton (Kr), neon (Ne), freon, dimethyl ether (DME), propylene, ethyl alcohol, methyl alcohol, nitrogen (N₂), dimethyl sulfide (DMS), isobutane, ethane, nitrous oxide (N₂O), silane (SiH₄), volatile organic compounds (VOCs), phosphine (PH₃), nitric oxide (NO), ethylene oxide, ethylbenzene, acetylene, and isopropyl alcohol, and mixtures thereof. Such target gases can be set variously according to the design and application field of the sensor, and can be adjusted and utilized according to specific conditions.

In the context of this specification, the meaning of "adsorption" of gas may include a process in which a material of the preconcentration unit attracts or binds a specific target gas to a surface through physical or chemical interactions. Physical adsorption may depend on intermolecular attractive forces, and chemical adsorption may involve chemical bonding. This process can play an important role in selectively preconcentrating low-concentration gases by the preconcentration unit and increasing local concentration around the sensor.

The role of the preconcentration unit is to selectively adsorb and concentrate target gases from the surrounding environment, thereby increasing the detection sensitivity of the sensing unit. The preconcentration unit may be designed using a porous structure or with materials having high affinity for specific gases, and has the possibility of efficiently preconcentrating gases through this and controlling their release when necessary.

The predetermined adsorption conditions refer to physical or chemical environmental states set for the preconcentration unit to effectively adsorb target gases. These conditions may include ranges in which gas molecules can stably bind on the surface characteristics or pore structure of the preconcentration material, and may be one or more of temperature, pressure, humidity, presence or absence of light irradiation, and surrounding gas composition.

The predetermined release conditions may refer to specific conditions required to release gases adsorbed by the preconcentration unit. For example, a certain temperature rise, pressure change, irradiation with light of a specific wavelength, or pH change may be included. These conditions are defined according to the design and purpose of the sensor and can help precisely control the gas release process.

Adsorption can be actively promoted by actively inducing adsorption conditions, and then release can be passively induced by canceling the adsorption conditions. Conversely, after the target gas is adsorbed by passive adsorption, release can be actively induced by actively inducing release conditions. Additionally, a method of stepwise combining adsorption and release using both conditions may also be considered. For example, initially both adsorption conditions and release conditions are suppressed to induce adsorption by natural diffusion, and then at a specific point, adsorption conditions are strengthened and release conditions are sequentially applied, allowing more active control of gas accumulation and desorption behavior.

The role of the sensing unit is to detect the target gas released from the preconcentration unit and convert the concentration or presence of the gas into electrical, chemical, or physical signals. The sensing unit may be composed of various sensing materials such as metal oxides, organic compounds, or nanomaterials, and can play an important role in increasing detection accuracy and selectivity.

The sensing unit may be in direct contact with the preconcentration unit or may be spaced apart by a predetermined distance. As long as the gas released from the preconcentration unit can reach the sensing unit by diffusion in a state of significantly high concentration, the positions of the sensing unit and the preconcentration unit are not particularly limited.

The preconcentration unit selectively adsorbs target gases from the surrounding environment while the adsorption conditions are satisfied, and additional adsorption may be limited in a state where the adsorption conditions are relaxed or released. Such operation can affect the concentration efficiency before the sensing stage by inducing the preconcentration unit to accumulate gases for a certain period of time. Particularly, in the section where adsorption conditions are maintained, a relatively high gas density can be formed inside the preconcentration unit even if the external gas concentration is low. Additionally, the preconcentration unit can concentrate gas in a state where the release conditions are not satisfied, and can locally increase the gas concentration around the sensing unit by releasing gas only when the release conditions are satisfied.

In one embodiment, the adsorption conditions or the release conditions may be one or more selected from temperature, pressure, light, and pH. That is, the role of the preconcentration control unit is to provide or control the necessary environment so that the preconcentration unit can satisfy or maintain predetermined adsorption conditions and/or release conditions. For example, the release process can be activated by applying heat to the preconcentration unit, providing a light source, or controlling pressure. Such control can contribute to increasing the stability and repeatability of the sensor. The preconcentration control unit may also control adsorption conditions rather than release conditions. For example, the preconcentration control unit may operate to change the conditions under which the preconcentration unit adsorbs gas over time. For example, it may be controlled in a manner that maintains gas adsorption by the preconcentration unit for a certain period of time and then limits or stops adsorption.

In one embodiment, the preconcentration control unit may be configured to include temperature in addition to time as an adsorption condition. For example, when external air conditions are higher or lower than room temperature, the preconcentration control unit may drive a heater and/or cooler adjacent to the preconcentration unit to maintain the temperature of the preconcentration unit at room temperature or a preset adsorption temperature. Such temperature control can help form a stable adsorption environment even in environments where the adsorption characteristics of the preconcentration material are rapidly degraded.

In another embodiment, when the adsorption condition is temperature, the preconcentration unit may operate to adsorb the target gas in a relatively low temperature range. For example, preconcentration materials such as MOF, Tenax TA, and activated carbon may have enhanced physical interactions with gas molecules at low temperatures, and the preconcentration control unit may maintain the temperature of the preconcentration unit in a range favorable for adsorption by deactivating a heater or using a cooling structure. Such temperature-based adsorption can help stable preconcentration even under conditions where external environmental temperature fluctuates.

In another embodiment, the preconcentration control unit may be configured to include gas flow conditions as adsorption conditions. By forming flow around the preconcentration unit using a micro fan, micro pump, or flow path structure during the preconcentration process, the frequency of contact between the preconcentration material and the target gas can be increased. Such flow control can help increase the adsorption rate compared to an adsorption method that relies on diffusion, or concentrate more gas within a limited time.

When the adsorption condition is pressure, the preconcentration unit may be configured to adsorb the target gas in a relatively high pressure environment. For example, in porous polymers or MOF-based materials, the density of gas molecules entering the pores may increase as pressure increases. In this case, the preconcentration control unit may control adsorption efficiency by increasing the local pressure around the preconcentration unit using a micro pump or valve structure.

When the adsorption condition is light, the preconcentration unit may operate to adsorb gas in a state where light of a specific wavelength is not irradiated. For example, materials such as TiO₂, ZnO, and CNT may have changed surface reactivity upon light irradiation, so in the adsorption stage, the light source may be blocked to induce gas molecules to stably bind to the surface. This method can help adsorption stability when using preconcentration materials with high photoreactivity.

When the adsorption condition is pH, the preconcentration unit may be configured to adsorb the target gas in a specific pH environment. For example, functionalized polymer or MOF surfaces may have changed surface charge states under acidic or basic conditions, and accordingly, binding force with specific gas molecules may be controlled. The preconcentration control unit may maintain the pH around the preconcentration unit in a range favorable for adsorption using a humid environment or chemical buffering materials.

Meanwhile, when the release condition is temperature, the preconcentration unit may operate to release the adsorbed target gas by heating. For example, CNT, metal oxide, and MOF-based preconcentration materials may have weakened binding force with gases as temperature rises, and the preconcentration control unit may increase the temperature of the preconcentration unit through a microheater or resistance heating structure. Such heat-based release can be utilized in configuring repetitive sensing cycles.

When the release condition is pressure, the preconcentration unit may release adsorbed gas when exceeding or falling below a specific pressure range. This can be achieved through physical or chemical changes using pressure, and for example, can operate in a manner that increases adsorption force inside the preconcentration unit in a high-pressure environment and desorbs adsorbed gas in a low-pressure environment.

When the release condition is light, the preconcentration unit may release adsorbed gas in response to light of a specific wavelength. For example, specific light sources such as ultraviolet (UV) or infrared (IR) may be used to weaken chemical bonds in the preconcentration unit or deliver thermal energy to desorb gas. Other possible non-limiting examples of light may include all electromagnetic waves including X-rays, radio waves, terahertz waves, laser light, pulsed light, broadband spectrum light, or monochromatic light tuned to specific wavelength bands.

Light as the release condition may be selected according to the gas to be concentrated. In this case, the wavelength of the irradiated light may be set to correspond to the characteristics of the preconcentration unit or the concentrated gas molecules. Some wavelengths may be absorbed by the sensing material or cause interference, but by selecting according to the absorption characteristics of the preconcentration material or adsorbed gas, such effects can be mitigated. For example, by utilizing a wavelength band that specific gas molecules strongly absorb, weakening of bonds or desorption can be more efficiently induced.

When the release condition is pH, the preconcentration unit may release gas according to pH changes in the surrounding environment. This may be based on the principle that the surface chemistry of the preconcentration unit changes with pH, thereby controlling the binding strength of adsorbed gases. For example, the preconcentration unit may be designed to selectively desorb specific gases under acidic (pH < 7) or basic (pH > 7) conditions.

In yet another embodiment, the preconcentration control unit may operate to control adsorption conditions by combining two or more of temperature, flow, and time. For example, at the beginning of adsorption, flow may be increased to induce rapid gas inflow, and then after a certain period of time elapses, flow may be decreased and temperature maintained to sustain a stable adsorption state.

Non-limiting examples of other available release conditions may include pressure change, light irradiation (ultraviolet, visible light, infrared), pH change, electrical stimulation, magnetic field, humidity, gas mixture ratio, catalytic reaction, chemical reaction, vibration, sound waves, specific gas concentration, solvent vapor, plasma generation, radiation (e.g., X-rays, gamma rays), electrochemical reaction, flow rate change, ion concentration, specific temperature gradient, specific pressure gradient, thermal shock, water vapor concentration, electromagnetic wave irradiation, saturation state of specific gas, physical shock, activation of gas adsorbent, viscosity change of fluid, or diffusion rate of gas. These conditions may be appropriately adjusted according to the design purpose and application field of the sensor.

For example, the preconcentration control unit may be adjacent to the preconcentration unit. As another example, the preconcentration control unit may not constitute a physical coupling relationship while being communicatively coupled to the preconcentration unit or the sensing unit. In this way, the preconcentration control unit may perform the role of delivering energy such as heat, light, electrical signals, etc. to the preconcentration unit, and the generation or control method of such energy may be implemented in various forms. Therefore, the preconcentration control unit does not necessarily need to be integrated on the same chip or substrate on which the preconcentration unit is formed. A structure in which energy is supplied or control signals are transmitted through a drive circuit or a separate control module arranged externally may also be considered.

In one embodiment, the release condition may be temperature. When the release condition is temperature, the preconcentration unit may release adsorbed gas when exceeding or falling below a specific temperature range. For example, when the preconcentration unit is heated, the interaction between the adsorbed gas and the surface of the preconcentration unit may be weakened, and gas may be released. Conversely, in a low-temperature environment, adsorption force may increase so that gas can be stably concentrated. Such temperature-based release conditions have the possibility of being utilized in applications where specific gases can be selectively released through temperature control or cycles can be set for continuous detection. This can usefully function in various fields such as environmental monitoring, chemical process control, or laboratory experiments.

The release condition may be time itself as needed, or may be another physical quantity that changes over time. For example, a stepwise method is possible in which gas is released for a predetermined time under a certain temperature condition, and then switched to another temperature condition to release again for a certain time. Additionally, a method of gradually releasing gas by continuously changing from one starting temperature to another target temperature over time may also be considered. Furthermore, a method of releasing gas while periodically and repeatedly changing two or more temperatures may also be applied. Such time-based release control can be flexibly set according to the bonding characteristics or release behavior of the concentrated gas.

To control these release conditions, a device that generates heat may be used, which can directly transfer heat to the preconcentration unit or induce release of adsorbed gas by controlling the ambient temperature. For example, low-power heaters, infrared heat sources, or electrical resistance heating devices may be used. These devices are capable of precise temperature control and can help increase the repeatability and reliability of the sensor. Through this, the possibility of maximizing the efficiency of the sensor in application fields sensitive to temperature changes can be provided.

In one embodiment, the adsorption conditions or the release conditions may be temperature. In one embodiment, the preconcentration control unit may adsorb or release the target gas by changing the temperature of the preconcentration unit. In one embodiment, the preconcentration control unit may release the target gas by applying heat to the preconcentration unit. By this, the interaction between the adsorbed gas and the surface of the preconcentration unit is weakened, and the gas can be efficiently desorbed. The method of applying heat may be implemented through low-power heaters, infrared heat sources, or electrical resistance heating devices, thereby providing the possibility of precisely controlling the release conditions. Such a heat-based release mechanism can contribute to improving the detection sensitivity of low-concentration gases and increasing sensing efficiency by selectively releasing preconcentrated gas when necessary. Furthermore, by supporting continuous and repetitive gas preconcentration and release processes, it also has the possibility of expanding the long-term reliability and application range of the sensor.

In one embodiment, the gas sensor may further comprise a flow control unit capable of controlling gas flow around the preconcentration unit and the sensing unit, controlling a size of a space through which gas can flow, or controlling opening and closing of a space through which gas can flow. The role of the flow control unit can control gas flow around the preconcentration unit and sensing unit and/or control the size or opening/closing of a space through which gas can diffuse, which can affect how effectively the preconcentration unit and/or sensing unit can form conditions for interacting with gas. Additionally, how efficiently the gas concentration locally increased by the preconcentration unit can be lowered can directly affect the response time and recovery time, which are important for the performance of the gas sensor. For example, the flow control unit may operate in a manner that opens a flow path with the external environment during the preconcentration stage so that gas flows smoothly into the preconcentration unit, and partially blocks the space around the sensor using a micro shutter, valve, or variable structure during the gas release stage. Accordingly, it is possible to reduce the phenomenon of released gas rapidly diffusing to the outside and control it to stay near the sensing unit for a certain period of time. Such flow control more effectively maintains local gas concentration and can also control response time reduction or recovery process.

In one embodiment, the gas sensor may further comprise a control unit that is at least communicatively coupled to the preconcentration unit, the sensing unit, and the preconcentration control unit, and controls the preconcentration control unit to satisfy or not satisfy the release conditions under predetermined control conditions. The control conditions are necessary for stable qualitative analysis or quantitative analysis.

In one embodiment, the control conditions may comprise elapse of a predetermined period of time. That is, by releasing preconcentrated gas at regular intervals and performing periodic sensing rather than continuous sensing, even low-concentration gases can be detected more effectively. This method can help reduce the energy consumption of the sensor and maintain gas detection sensitivity. When the preconcentration unit concentrates gas for a certain period of time and then releases it, the sensing unit detects a locally high concentration of gas, which has the possibility of lowering the detection limit. Additionally, periodic sensing can provide the advantage of reducing the burden of data analysis and storage and extending the life of the sensor in environments requiring long-term monitoring.

In one embodiment, the preconcentration unit and/or sensing unit may comprise one or more selected from: porous polymers or composites thereof including Tenax TA and Tenax GR; carbon allotropes or conductive nanomaterials selected from aligned or non-aligned carbon nanotubes (CNT), graphene, graphene oxide, activated carbon, and activated carbon fiber (ACF); metal-organic frameworks (MOF) or covalent organic frameworks (COF) including ZIF-8, ZIF-67, UiO-66, and MIL series; and metal oxide-based materials selected from SnO₂, ZnO, TiO₂, and WO₃. For example, the preconcentration unit and/or sensing unit may include one or more of Tenax TA, CNT, metal oxides, activated carbon, activated charcoal, or MOF.

As a preconcentration material, Tenax TA is a polymer-based adsorbent primarily used to selectively concentrate gases with nonpolar or weak polar characteristics such as volatile organic compounds (VOCs). Tenax TA has high thermal stability and has the characteristic of maintaining a low background signal during gas adsorption and desorption processes. These characteristics increase the reliability of the sensor and enable precise gas detection. Additionally, Tenax TA provides a large surface area through a porous structure and can effectively concentrate low-concentration gases to improve sensing sensitivity.

As a preconcentration material, CNT is a nanostructure with a large surface area and excellent chemical stability, and has the characteristic of effectively preconcentrating various gases. CNT mainly has excellent conductivity and adsorption characteristics, and provides a functionalized surface that can form strong interactions with specific gas molecules. These characteristics increase gas detection sensitivity and enable concentration and selective preconcentration of low-concentration gases.

Metal oxides are compounds in which oxygen and metal atoms are bonded, and generally include materials such as SnO₂, ZnO, TiO₂, and WO₃. These materials show the characteristic that electrical properties (e.g., resistance) change by reacting with specific gases, and can be utilized for detection of low-concentration gases.

MOF is an abbreviation for Metal-Organic Framework, and is a material with a porous structure formed by bonding metal ions and organic ligands. MOF can selectively adsorb various gases due to its high surface area and variable chemical properties, and has the possibility of being used as a multi-purpose gas sensing material.

As a preconcentration material, MOF (Metal-Organic Framework) has excellent gas adsorption and selective concentration capabilities. MOF provides a very large surface area and controllable pore size, enabling selective concentration of gases with various sizes and characteristics. Additionally, MOF has high chemical and thermal stability, so performance can be maintained even during repetitive gas adsorption and desorption processes. In particular, MOF can optimize interaction with specific gases through the combination of metal centers and organic ligands, and can provide high selectivity and sensitivity for target gases.

Other non-limiting examples of sensing materials include graphene, silica nanoparticles, polyaniline, perovskite, mesoporous silica, boron nitride, titanium dioxide (TiO₂), fluorinated carbon, ceramic composites, plasmonic nanoparticles, polythiophene, organic semiconductors, heterometallic nanoclusters, fullerene, silver nanoparticles, gold nanoparticles, carbon black, organic-inorganic hybrids, bio-based nanoparticles, ionic liquids, zeolite, cuprite (Cu₂O), vanadium oxide, carbon quantum dots, gallium nitride (GaN), porous carbon, diamond nanoparticles, nickel oxide (NiO), molybdenum disulfide (MoS₂), tungsten disulfide (WS₂), cellulose nanofibers, silica aerogel, nano copper oxide, PANI composites, manganese dioxide (MnO₂), ferrite, silver nanoclusters, optical dyes, porphyrin-based compounds, photocatalytic materials, plasma-activated materials, and heat-treated oxides. These materials can provide various options for controlling the sensitivity, selectivity, and response speed of sensors.

In one embodiment, the gas sensor may further comprise a signal analysis unit that is at least communicatively coupled to the sensing unit and configured to analyze signals generated by the sensing unit. An example of signals generated by the sensing unit is an electrochemical signal, in which a specific gas reacts with the sensing unit to generate a change in current, voltage, or resistance. This method provides the possibility of precisely detecting gas concentration and is particularly advantageous for increasing sensitivity in low-concentration gas detection.

Another example of a method by which the sensing unit generates a signal is a catalytic combustion method, in which gas causes an oxidation reaction on a catalyst surface to generate heat and electrical signals. This method is primarily suitable for detection of flammable gases and can operate stably in highly reactive environments.

Another example of a method by which the sensing unit generates a signal is a mechanical resonance method, which utilizes the principle that specific gases adsorb or desorb on a resonator surface, causing changes in resonance frequency. This method is sensitive to mass changes of gases and has potential for utilization in applications requiring precise concentration analysis.

Another example of a method by which the sensing unit generates a signal is optical sensing, which measures characteristics of light of specific wavelengths being absorbed, reflected, or scattered through interaction with gas. This method is advantageous for non-contact detection and has the possibility of providing high reliability even under various environmental conditions.

The analysis method of the signal analysis unit is determined according to signals generated by the sensing unit, and other non-limiting examples of sensing unit operation methods may include infrared absorption method, surface plasmon resonance method, solution conductivity change, thermal conductivity change, electromagnetic wave reflection, surface response spectroscopy, optical fiber sensing, surface nanostructure-based light absorption, color change sensor, photothermal conduction method, Raman spectroscopy, micro heat transfer, liquid chromatography-based method, microgravity sensing, acoustic wave detection, resonator-based electromagnetic wave detection, elastic wave-based sensing, quantum dot-based optical detection, nano-optical-based absorption analysis, ionic conductivity change, nanotube-based electrochemistry, fluorescence spectroscopy, porous structure-based absorption, thermoluminescence analysis after gas adsorption, and electron spin resonance method. These methods can be selected and applied to suit various application environments and target gases.

The advantage of the gas sensor of the present invention described above lies in providing the possibility of effectively detecting low-concentration target gases in various environments. Through the integrated design of the preconcentration unit and sensing unit, miniaturization and simplification of the system are possible, thereby increasing portability and ease of installation. Additionally, through selection of various preconcentration materials and sensing materials, high selectivity for specific gases can be provided, thereby enabling more accurate detection of trace amounts of gas that are difficult to detect with conventional sensors. The concentration function of the preconcentration unit and precise control of the preconcentration control unit can contribute to improving the sensitivity and detection limit of the sensor. Design considering energy efficiency and repeatability supports long-term monitoring and sustainable operation, and can expand utilization possibilities in various application fields such as environmental monitoring, industrial safety, and medical diagnosis. These features show the possibility that the present invention can overcome the limitations of existing technology and present a standard for new gas detection technology.

Meanwhile, a low-concentration gas sensing method according to an embodiment of the present invention may comprise steps of preparing a gas sensor and sensing, through a sensing unit of the gas sensor, a target gas that a preconcentration unit of the gas sensor releases after adsorption. By sensing gas as described above, the possibility of effectively detecting low-concentration target gases that are difficult to detect by conventional methods can be provided. By having the preconcentration unit adsorb gas and release concentrated gas under certain conditions, the sensing unit can detect locally increased gas concentration to increase detection sensitivity. This method is suitable for qualitative and quantitative analysis of trace gases, and can be particularly useful in application fields such as hazardous gas detection in environmental monitoring or industrial sites. Additionally, by repeatedly performing preconcentration and release processes, continuous data collection is possible, and through this, advantages can be had in being able to detect changes in gas concentration over time or abnormal states in real time.

In one embodiment, the gas sensor may comprise: a preconcentration unit comprising a material that adsorbs a predetermined target gas, and configured to adsorb the target gas under predetermined adsorption conditions and/or release the adsorbed target gas under predetermined release conditions; a sensing unit adjacent to the preconcentration unit and configured to sense at least the target gas released by the preconcentration unit; and a preconcentration control unit configured to control satisfaction or non-satisfaction of the adsorption conditions and/or the release conditions.

In one embodiment, the adsorption conditions or the release conditions may be provided to comprise one or more selected from temperature, pressure, light, and pH. In one embodiment, the adsorption conditions or the release conditions may be temperature. In one embodiment, the preconcentration control unit may be configured to adsorb or release the target gas by changing the temperature of the preconcentration unit. In one embodiment, the preconcentration control unit may release the target gas by applying heat to the preconcentration unit. In one embodiment, the gas sensor may be provided to further comprise a control unit that is at least communicatively coupled to the preconcentration unit, the sensing unit, and the preconcentration control unit, and controls the preconcentration control unit to satisfy or not satisfy the adsorption conditions and/or the release conditions under predetermined control conditions. In one embodiment, the control unit may be provided such that the control conditions are satisfied when a predetermined period of time elapses. In one embodiment, the preconcentration unit or the sensing unit may be provided to comprise one or more selected from: porous polymers or composites thereof including Tenax TA and Tenax GR; carbon allotropes or conductive nanomaterials selected from aligned or non-aligned carbon nanotubes (CNT), graphene, graphene oxide, activated carbon, and activated carbon fiber (ACF); metal-organic frameworks (MOF) or covalent organic frameworks (COF) including ZIF-8, ZIF-67, UiO-66, and MIL series; and metal oxide-based materials selected from SnO₂, ZnO, TiO₂, and WO₃. In one embodiment, the preconcentration unit and/or sensing unit may be provided to comprise one or more of Tenax TA, CNT, metal oxides, activated charcoal, activated carbon, or MOF. In one embodiment, the gas sensor may be provided to further comprise a signal analysis unit that is at least communicatively coupled to the sensing unit and configured to analyze signals generated by the sensing unit.

The advantage of the gas sensing method of the present invention described above lies in providing the possibility of effectively detecting low-concentration target gases. By concentrating and releasing gas through the preconcentration unit so that the sensing unit can detect gas at higher concentrations, detection sensitivity and accuracy can be greatly improved. This method is more energy efficient compared to conventional continuous detection methods and has the possibility of extending the life of the sensor through periodic sensing processes. Additionally, through combinations of various release conditions and sensing materials, various types of gases can be selectively detected, providing a wide range of applications. This method can be useful in various fields such as environmental monitoring, industrial safety, and medical diagnosis, and has the potential to overcome the limitations of existing gas sensing technology and present new standards.

Meanwhile, gas sensors or gas sensing methods according to embodiments of the present invention can be used in various applications. In one embodiment, the present invention provides a gas monitoring device comprising: a gas sensor comprising: a preconcentration unit capable of adsorbing a predetermined target gas and releasing the adsorbed target gas under predetermined release conditions; a sensing unit adjacent to the preconcentration unit and configured to sense at least the target gas released by the preconcentration unit; and a preconcentration control unit capable of controlling satisfaction or non-satisfaction of predetermined release conditions under which the preconcentration unit releases the target gas; and a signal generation unit configured to generate a signal when the gas sensor detects the target gas at or above a predetermined level.

A gas monitoring device refers to a device capable of performing the role of detecting the concentration of target gas in real time in a specific environment and generating alarms or signals according to detected information to notify users or systems. Such a device can concentrate specific gas through a preconcentration unit, have a sensing unit detect the concentrated gas, and output a warning signal through a signal generation unit when exceeding established criteria. Gas monitoring devices have the possibility of being utilized in various fields such as environmental monitoring, industrial safety, medical diagnosis, and household air quality management. In particular, by early detection of hazardous gases or low concentrations of dangerous substances, accidents can be prevented and opportunities can be provided for users to take prompt action.

In one embodiment, the gas monitoring device may be an air pollution detection device, a hazardous gas detection device, an industrial emission detection device, an indoor air quality monitoring device, a chemical plant gas leak alarm device, a household CO₂ monitoring device, a portable radon detector, a medical breath analysis device, an agricultural greenhouse gas detection device, an atmospheric methane detection device, a vehicle exhaust gas detection device, a refinery process gas analysis device, a semiconductor manufacturing process gas monitoring device, a wastewater treatment process gas detection device, a food storage ethylene detection device, a cold storage ammonia detection device, a fire detection smoke analysis device, a radioactive leak detection device, a building carbon dioxide concentration measurement device, an oxygen deficiency alarm device, an anesthetic gas concentration monitoring device, a precision gas chromatography device, a gas fuel mixture ratio measurement device, an offshore oil platform gas detection device, a military chemical warfare response gas detector, a spacecraft internal air quality monitoring device, a natural gas distribution network detection device, an underground mine methane detection device, an electric vehicle battery overheating detection device, a laboratory toxic gas monitoring device, a refrigerant leak detection device, a plasma process gas detection device, an incubator internal oxygen and CO₂ concentration detection device, a power plant exhaust gas detection device, a hydrogen fuel cell system hydrogen leak detection device, an earthquake precursor gas detection device, a city gas supply network detection device, a medical helium gas concentration detection device, a high-altitude oxygen concentration monitoring device, a smart building VOC detection device, a crop storage facility carbon dioxide detection device, a hospital waste incinerator gas monitoring device, an aircraft cabin air quality monitoring device, a combustion process NOx and SOx detection device, an energy plant gas detection device, a subway tunnel air quality detection device, an undersea gas leak detection device, a smart city environmental sensor network, or a biological laboratory gas monitoring device.

The advantage of applications such as the gas monitoring device according to embodiments of the present invention lies in being able to detect gas in real time in various environments and issue alarms quickly. This device provides the possibility of improving detection sensitivity and accuracy compared to conventional methods through the function of selectively concentrating and detecting target gas. Additionally, it is suitable for low-concentration gas detection and is energy efficient and capable of long-term use by periodically performing preconcentration and release processes. By providing immediate notification when gas exceeding reference values is detected through the signal generation unit, practical help can be provided in preventing accidents or environmental pollution. Furthermore, it can be expanded for multi-purpose use by combining various sensing materials and release conditions, and has the potential to be practically utilized in various application fields such as industrial safety, environmental monitoring, and medical diagnosis. Such devices can satisfy both safety and efficiency by providing users with more reliable data and flexibility.

Hereinafter, embodiments of the present invention will be described. However, the embodiments described below are merely some embodiments of the present invention, and the scope of the present invention is not limited to the following embodiments.

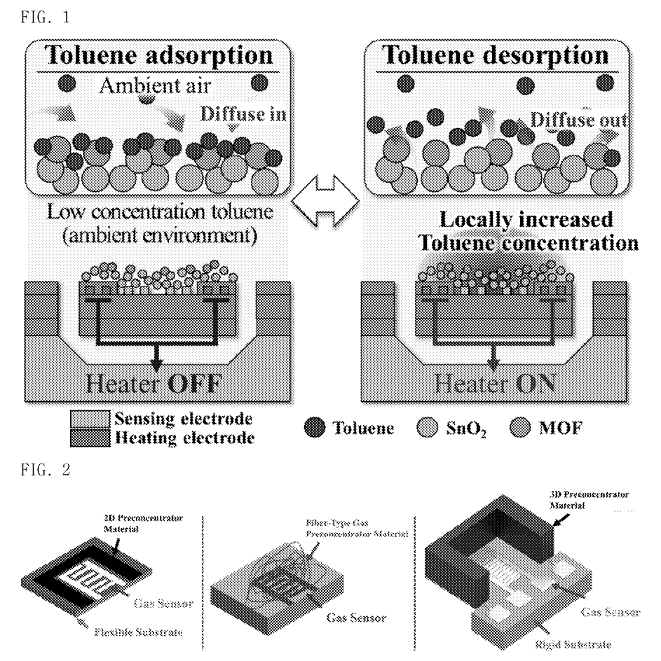

FIG. 1 is a diagram illustrating the adsorption and desorption process of toluene gas. This diagram explains the operating principle of a gas sensor in which a preconcentration unit and a sensing unit are integrated. Referring to FIG. 1, the preconcentration unit is composed of MOF and SnO₂ materials and adsorbs low-concentration toluene gas, showing a process in which toluene present in ambient air is adsorbed and concentrated on the MOF and SnO₂ surfaces in a state where the heater is off (Heater OFF). Conversely, in a state where the heater is on (Heater ON), it shows a process in which the preconcentration unit is heated, the adsorbed toluene is desorbed to generate locally high concentration toluene, and the sensing unit detects this. This process occurs through diffusion of gas ("Diffuse in" during adsorption, "Diffuse out" during desorption). Through FIG. 1, it can be confirmed that the gas sensor of the present invention can improve detection sensitivity by concentrating low-concentration gas and lower the detection limit by generating locally high concentration.

In particular, FIG. 1 is a schematic diagram of a gas sensor with an integrated preconcentrator, showing the core operating principle of this system. When low-concentration gas exists in the surrounding environment, the preconcentrator chemically or physically adsorbs it. Subsequently, when energy is applied such as raising temperature or irradiating light through a device integrated in the system or an external device, the gas adsorbed to the preconcentrator is desorbed. As a result, the gas concentration around the sensor becomes locally higher than the surrounding environment for a certain period of time. By having the gas sensing material of the sensor detect this concentrated gas, the detection sensitivity of the sensor can be improved and the detection limit can be lowered. Through this process, the possibility of more effectively detecting low-concentration gas is provided.

Looking at the components of a gas sensor with an integrated preconcentrator, selection of preconcentration material plays an important role. Materials that can effectively concentrate gas include Tenax TA, CNT (carbon nanotubes), Metal Organic Frameworks (MOF), etc. These materials provide the ability to selectively adsorb specific gases through their respective unique characteristics. These characteristics contribute to improving the detection accuracy and selectivity of the sensor, and present the possibility of effectively detecting various gases through appropriate combinations of not only sensing materials but also preconcentration materials.

Gas sensing materials use materials that induce physical or chemical changes when in contact with gas. Representatively, metal oxide corresponds to this, and this material causes changes in electrical properties by reacting with specific gases. Such characteristic changes can be utilized to detect the presence and concentration of gas, and can contribute to increasing detection sensitivity and accuracy. Metal oxide is a widely used material in the gas sensing field and has the potential to provide highly reliable data in various application environments.

FIG. 2 is a diagram illustrating various forms of gas preconcentration materials and the structure of gas sensors utilizing them. This diagram explains examples in which 2D preconcentration material, fiber-type preconcentration material, and 3D preconcentration material are each combined with gas sensors. Referring to FIG. 2, the left diagram shows a structure in which 2D preconcentration material is arranged on a flexible substrate, showing the possibility of applying the sensor in various forms using a flexible substrate. The center diagram shows a structure in which fiber-type gas preconcentration material is combined with a sensor, playing a role of increasing gas preconcentration efficiency through porous and fiber structures. The right diagram shows a structure in which 3D preconcentration material is applied on a rigid substrate, showing a structure capable of effectively concentrating and detecting high concentrations of gas using three-dimensional preconcentration material. Through FIG. 2, it can be confirmed that the gas sensor of the present invention can adapt to various environments and application fields by combining the form of preconcentration material and characteristics of substrate.

As a structural characteristic of the preconcentrator, it may have a 3D form, and cases coated in 2D form are also possible as needed. One of the important characteristics appearing in these various structural characteristics is that porous materials can form narrow channels. This operates on a principle similar to molecular sieves, and can selectively control the types of gas delivered to the gas sensing material by utilizing differences in diffusion coefficients according to the size of concentrated gas. Additionally, when the amount of adsorbed gas is saturated, the narrower the channel size, the more the average gas concentration inside the channel increases during desorption, which can help increase detection sensitivity. Furthermore, by reducing the phenomenon of desorbed gas being pushed out by external flow, the advantage can also be provided that high gas concentration around the gas sensing material can be maintained for a long time. These structural characteristics show the possibility of providing flexibility and adaptability in various application fields.

FIG. 3 is a diagram illustrating the structure of a gas sensor platform and the arrangement of a preconcentrator and gas sensing material. This diagram visually explains the components of the sensor and the gas detection and concentration functions. Referring to FIG. 3, the upper left diagram shows the structure of the sensor platform, showing a configuration in which a microheater and sensing electrodes are arranged. This structure includes regions coated with gas sensing material and preconcentration material. The upper right diagram shows, through an SEM image, a magnified view of the region coated with the preconcentrator and gas sensing material. The lower left diagram and lower right diagram are images showing the distribution of gas sensing material and preconcentrator material, respectively, confirming that each material is uniformly coated. Through FIG. 3, it can be confirmed that the gas sensor platform of the present invention has structural advantages of being able to precisely and uniformly arrange the preconcentrator and gas sensing material. This design can improve gas detection sensitivity and selectivity, and shows that efficient gas concentration and detection are possible through precise temperature control using a microheater. This platform provides the possibility of supporting highly reliable gas sensing in various application environments. FIG. 3 shows an example of a gas sensor utilizing a MEMS chip as one of various gas sensor structures. The gas sensor platform of the present invention is configured by coating gas sensing material and gas preconcentration material on a MEMS chip, thereby enabling efficient and precise gas sensor fabrication. This structure provides a foundation for simultaneously implementing miniaturization and high performance by utilizing the advantages of MEMS technology.

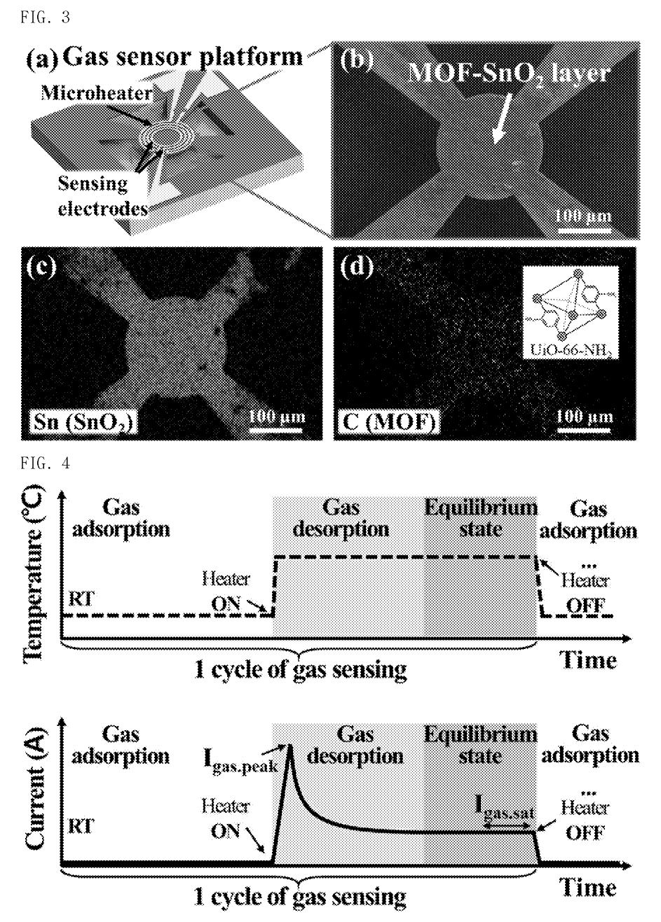

FIG. 4 is a diagram illustrating temperature changes and gas signal changes during one cycle in which the gas sensor of the present invention detects gas. The upper diagram of FIG. 4 shows temperature changes during one cycle, and the lower diagram of FIG. 4 shows changes in gas signal during one cycle. This diagram visually explains the process leading from gas adsorption, desorption, and back to adsorption. Referring to FIG. 4, initially, in a state where the heater is off (Heater OFF), a step proceeds in which the preconcentration unit adsorbs gas at low temperature (RT, room temperature). Subsequently, when the temperature is raised through the heater and the preconcentration unit is activated, the adsorbed gas is desorbed, and accordingly the gas signal of the sensor reaches a maximum value (maximum gas signal). After gas desorption, the heater is turned off again, the preconcentration unit returns to low temperature, and adsorption of the next cycle begins. Through FIG. 4, it can be confirmed that the gas sensor of the present invention can increase gas detection sensitivity and provide stable signals through periodic temperature control and concentration-desorption processes. This cyclic structure is suitable for low-concentration gas detection and repetitive data collection, and shows the possibility of supporting long-term detection in an energy-efficient manner. Additionally, it can be confirmed that accurate gas concentration analysis is possible by utilizing quantitative data such as maximum gas signal.

Another important point that can be confirmed through FIG. 4 is the possibility of more precisely analyzing gas concentration and type by comparing the maximum gas signal and saturated gas signal (Isat). The saturated gas signal is a signal measured in a state where the gas concentration of the surrounding environment is stabilized and the surroundings of the sensing material have the same gas composition as the external environment, which reflects the influence of all gas unrelated to preconcentration. On the other hand, the maximum gas signal is mainly composed of signals from "predetermined target gas" selectively concentrated according to the characteristics of the preconcentration material. By utilizing this difference, not only can the concentration of target gas be predicted more precisely, but there also exists the possibility of optimizing the configuration of sensor arrays to reduce the number of sensors needed. This can contribute to improving detection efficiency in complex gas environments and reducing design costs and resources of sensor systems.

FIG. 4 shows the gas detection process of a gas sensor with an integrated preconcentrator. The sensor concentrates gas from the surrounding environment for a set time, and then goes through a step of desorbing the concentrated gas along with activation of the gas sensing material. The concentration of target gas can be predicted by analyzing the maximum gas signal generated in this process. When the gas signal decreases due to desorbed gas, the sensor repeats the process of concentrating gas again. At each step, the detection process can be optimized by controlling or stopping gas supply when necessary. This periodic process can support stable gas detection and accurate concentration analysis.

The preconcentration unit of the present invention can selectively adsorb various types of gases, and the gases subject to adsorption include the following representative gas groups. Volatile organic compounds (VOCs) include benzene, toluene, xylene, formaldehyde, acetone, ethanol, etc., and reactive or hazardous gases may include ammonia (NH₃), hydrogen sulfide (H₂S), nitrogen oxides (NOx), sulfur oxides (SOx), carbon monoxide (CO), ozone (O₃), etc. Additionally, hydrogen (H₂), carbon dioxide (CO₂), and moisture (H₂O), which require detection in general hazardous environments or industrial processes, may also be included in target gases processable by the preconcentration unit.

The preconcentration unit according to the present invention basically includes thermal desorption-based operation, and various forms of energy supply methods can be applied for this purpose. For example, a direct heating method (resistance heating, Joule heating) using a microheater formed below or inside the preconcentration unit may be applied. Additionally, it is possible to radiatively heat the preconcentration unit in a non-contact manner using IR lamps, lasers, halogen light sources, etc. Additionally, induction heating methods that form a metal conductive layer at the bottom of the concentration layer and locally raise temperature by applying RF or microwave (MW) are also feasible.

The preconcentration unit can operate in a temperature programming manner, preferentially releasing physically adsorbed gases (H₂O, low molecular weight VOC, etc.) at low temperatures, and enabling stepwise desorption that releases gases with strong chemisorption forces such as ammonia or nitrogen oxides at high temperatures. Through such temperature profile control, the timing at which specific gases are dominantly released can be selectively controlled, which can help improve gas sensing sensitivity. In addition, photodesorption methods in which surface bonding is weakened in MOF, CNT, metal oxides, etc. by irradiating UV or visible light, or methods of releasing gas by reducing binding force based on electrostatic energy can be used together.

The preconcentration unit of the present invention can be implemented in various types according to various materials and structural characteristics, and the following are representative embodiments by preconcentration material group.

### Group A. Porous Polymers and Composites Thereof

In one embodiment, the preconcentration unit may include porous polymer-based materials such as Tenax TA, Tenax GR, etc. These materials can be manufactured through processes such as spray coating, freeze casting, electrospinning, electrospraying, etc., and have high adsorption characteristics for VOCs. Polymer-based preconcentration units can be controlled so that desorption occurs at approximately 300°C or below in consideration of thermal stability, thereby maintaining structural stability during repetitive adsorption-desorption processes.

### Group B. Carbon Allotropes and Conductive Nanomaterials

In another embodiment, the preconcentration unit may include CNT (aligned or non-aligned form), graphene or graphene oxide, activated carbon, activated carbon fiber (ACF), etc. These materials exhibit excellent adsorption characteristics for VOCs and odorous gases (H₂S, etc.) based on large specific surface area and high chemical stability. These materials with conductive characteristics can implement thermal desorption by self-heating in which the material itself generates heat by applying current to both ends, and induction heating methods using microwave (MW) or RF are also possible. Additionally, these nanostructures can activate photodesorption in response to light irradiation in a wide wavelength range.

### Group C. Porous Crystalline Structures (MOFs and COFs)

In yet another embodiment, the preconcentration unit may include various MOF materials such as ZIF-8,

ZIF-67, UiO-66, MIL series, etc., or COF (Covalent Organic Framework) materials. Since these materials have structures in which pore size and chemical bonding environment are precisely designed, selective concentration is possible for gases with specific size and bonding characteristics such as low molecular weight VOC, amines (NH₃, etc.), CO₂, etc. For MOF and COF, it is preferable to perform thermal desorption at relatively low temperatures (approximately 300°C or below) to prevent structural collapse, and photodesorption methods using UV light sources can also be applied together.

### Group D. Metal Oxides

In yet another embodiment, the preconcentration unit may include metal oxide-based materials such as SnO₂, ZnO, TiO₂, WO₃, etc. These materials are sensitive to surface charge changes and can effectively adsorb polar or reactive gases such as NH₃, NOx, SOx, O₃, etc. Metal oxide-based preconcentration units can apply high-temperature heating of 300°C or higher to dissociate surface chemical bonds, and have the characteristic that photodesorption activity increases as oxygen vacancy changes occur through UV irradiation. By utilizing these characteristics, selective release of specific gases and improvement of detection sensitivity are possible.

These various embodiments allow flexible design so that the preconcentrator-integrated gas sensor of the present invention can respond to various environmental conditions and target gases for detection, and can provide gas sensing performance with high sensitivity, selectivity, and repeatability through combinations of preconcentration materials and desorption mechanisms.

Although the present invention has been described above with reference to preferred embodiments, those skilled in the art will understand that various modifications and changes can be made to the present invention within the scope not departing from the spirit and scope of the present invention as set forth in the following claims.

What is claimed is:

1. A gas sensor comprising: a preconcentration unit comprising a material that adsorbs a predetermined target gas, and configured to adsorb the target gas under predetermined adsorption conditions and/or release the adsorbed target gas under predetermined release conditions; a sensing unit adjacent to the preconcentration unit and configured to sense at least the target gas released by the preconcentration unit; and a preconcentration control unit configured to control satisfaction or non-satisfaction of the adsorption conditions and/or the release conditions.

2. The gas sensor of claim 1, wherein the adsorption conditions or the release conditions comprise one or more selected from temperature, pressure, light, and pH.

3. The gas sensor of claim 2, wherein the adsorption conditions or the release conditions comprise temperature, and the preconcentration control unit is configured to change the temperature of the preconcentration unit to adsorb or release the target gas.

4. The gas sensor of claim 1, further comprising a control unit that is at least communicatively coupled to the preconcentration unit, the sensing unit, and the preconcentration control unit, and is configured to command the preconcentration control unit to satisfy or not satisfy the adsorption conditions and/or the release conditions under predetermined control conditions.

5. The gas sensor of claim 4, wherein the control conditions comprise elapse of a predetermined period of time.

6. The gas sensor of claim 1, wherein the preconcentration unit or the sensing unit comprises one or more selected from: porous polymers or composites thereof including Tenax TA and Tenax GR; carbon allotropes or conductive nanomaterials selected from aligned or non-aligned carbon nanotubes (CNT), graphene, graphene oxide, activated carbon, and activated carbon fiber (ACF); metal-organic frameworks (MOF) or covalent organic frameworks (COF) including ZIF-8, ZIF-67, UiO-66, and MIL series; and metal oxide-based materials selected from SnO₂, ZnO, TiO₂, and WO₃.

7. The gas sensor of claim 1, further comprising a signal analysis unit that is at least communicatively coupled to the sensing unit and configured to analyze signals generated by the sensing unit.

8. The gas sensor of claim 1, further comprising a flow control unit capable of controlling gas flow around the preconcentration unit and the sensing unit, controlling a size of a space through which gas can flow, or controlling opening and closing of a space through which gas can flow.

9. A low-concentration gas sensing method comprising: providing a gas sensor comprising: a preconcentration unit comprising a material that adsorbs a predetermined target gas, and configured to adsorb the target gas under predetermined adsorption conditions and/or release the adsorbed target gas under predetermined release conditions; a sensing unit adjacent to the preconcentration unit and configured to sense at least the target gas released by the preconcentration unit; and a preconcentration control unit configured to control satisfaction or non-satisfaction of the adsorption conditions and/or the release conditions; and sensing, through the sensing unit, the target gas that the preconcentration unit releases after adsorption.

10. The method of claim 9, wherein the adsorption conditions or the release conditions are provided to comprise one or more selected from temperature, pressure, light, and pH.

11. The method of claim 10, wherein the adsorption conditions or the release conditions are provided to comprise temperature, and the preconcentration control unit is configured to change the temperature of the preconcentration unit to adsorb or release the target gas.

12. The method of claim 9, wherein the gas sensor is provided to further comprise a control unit that is at least communicatively coupled to the preconcentration unit, the sensing unit, and the preconcentration control unit, and is configured to command the preconcentration control unit to satisfy or not satisfy the adsorption conditions and/or the release conditions under predetermined control conditions.

13. The method of claim 12, wherein the control unit is provided such that the control conditions are satisfied when a predetermined period of time elapses.

14. The method of claim 9, wherein the preconcentration unit or the sensing unit comprises one or more selected from: porous polymers or composites thereof including Tenax TA and Tenax GR; carbon allotropes or conductive nanomaterials selected from aligned or non-aligned carbon nanotubes (CNT), graphene, graphene oxide, activated carbon, and activated carbon fiber (ACF); metal-organic frameworks (MOF) or covalent organic frameworks (COF) including ZIF-8, ZIF-67, UiO-66, and MIL series; and metal oxide-based materials selected from SnO₂, ZnO, TiO₂, and WO₃.

15. The method of claim 9, wherein the gas sensor is provided to further comprise a signal analysis unit that is at least communicatively coupled to the sensing unit and configured to analyze signals generated by the sensing unit.

16. The method of claim 9, wherein the gas sensor is provided to further comprise a flow control unit capable of controlling gas flow around the preconcentration unit and the sensing unit, controlling a size of a space through which gas can flow, or controlling opening and closing of a space through which gas can flow.

17. A gas monitoring device comprising: a gas sensor comprising: a preconcentration unit comprising a material that adsorbs a predetermined target gas, and configured to adsorb the target gas under predetermined adsorption conditions and/or release the adsorbed target gas under predetermined release conditions; a sensing unit adjacent to the preconcentration unit and configured to sense at least the target gas released by the preconcentration unit; and a preconcentration control unit configured to control satisfaction or non-satisfaction of the adsorption conditions and/or the release conditions; and a signal generation unit configured to generate a signal when the gas sensor detects the target gas at or above a predetermined level.
